(19) United States
(12) Patent Application Publication
(10) Pub. No.: US 2023/0241302 A1
(43) Pub. Date: Aug. 3, 2023

(54) ASPIRATION CATHETERS HAVING GROOVED INNER SURFACES, AND ASSOCIATED SYSTEMS AND METHODS

(71) Applicant: Inari Medical, Inc., Irvine, CA (US)

(72) Inventors: Benjamin Edward Merritt, San Clemente, CA (US); Garrett Thomas Offerman, Orange, CA (US); Kali Wen-Tseng Slaughter, Santa Ana, CA (US); John Coleman Thress, Capistrano Beach, CA (US); Brian Michael Strauss, San Clemente, CA (US); Donald Joseph Fuller, San Clemente, CA (US); Jared Shimizu, Tustin, CA (US)

(21) Appl. No.: 18/159,507

(22) Filed: Jan. 25, 2023

Related U.S. Application Data
(60) Provisional application No. 63/304,748, filed on Jan. 31, 2022.
(60) Provisional application No. 63/395,586, filed on Aug. 5, 2022.

(51) Int. Cl. A61M 1/00

(52) U.S. Cl. CPC A61M 1/84; A61M 1/71

(57) ABSTRACT

Disclosed herein are aspiration catheters having grooved inner surfaces for removing clot material from the vasculature of a patient, and associated systems and methods. In some embodiments, an aspiration catheter can include a proximal terminus, a distal terminus, and an inner surface defining a lumen. The inner surface includes at least one groove formed therein and that extends from the distal terminus at least partially toward the proximal terminus. When clot material is aspirated through the aspiration catheter, the at least one groove can provide a leak path past the clot material to improve the ingestion of the clot material through the catheter and inhibit clogging.

### CROSS-REFERENCE TO RELATED APPLICATIONS

This application claims the benefit of U.S. Provisional Patent Application No. 63/304,748, filed Jan. 31, 2022, and titled “ASPIRATION CATHETERS HAVING GROOVED INNER SURFACES, AND ASSOCIATED SYSTEMS AND METHODS,” and U.S. Provisional Patent Application No. 63/395,586, filed Aug. 5, 2022, and titled “ASPIRATION CATHETERS HAVING GROOVED INNER SURFACES, AND ASSOCIATED SYSTEMS AND METHODS,” each of which is incorporated by reference herein in its entirety.

### TECHNICAL FIELD

The present technology generally relates to clot treatment systems including aspiration catheters having grooved or rifled inner surfaces to facilitate increased clot ingestion into the aspiration catheter, reduce clogging of the aspiration catheter, increase/optimize flowrate within the aspiration catheter, and/or enhance clot removal through the aspiration catheter.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. Infarction does not develop or is greatly limited if the flow of blood is reestablished rapidly. Failure to reestablish blood flow can lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT arises most commonly when there is a propensity for stagnated blood (e.g., long distance air travel, immobility, etc.) and clotting (e.g., cancer, recent surgery, such as orthopedic surgery, etc.). DVT causes harm by: (1) obstructing drainage of venous blood from the legs leading to swelling, ulcers, pain, and infection, and (2) serving as a reservoir for blood clots to travel to other parts of the body including the heart, lungs, brain (stroke), abdominal organs, and/or extremities.

In the pulmonary circulation, the undesirable material can cause harm by obstructing pulmonary arteries—a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

There are many existing techniques to reestablish blood flow through an occluded vessel. One common surgical technique, an embolectomy, involves incising a blood vessel and introducing a balloon-tipped device (such as the Fogarty catheter) to the location of the occlusion. The balloon is then inflated at a point beyond the clot and used to translate the obstructing material back to the point of incision. The obstructing material is then removed by the surgeon. Although such surgical techniques have been useful, exposing a patient to surgery may be traumatic and best avoided when possible. Additionally, the use of a Fogarty catheter may be problematic due to the possible risk of damaging the interior lining of the vessel as the catheter is being withdrawn.

Percutaneous methods are also utilized for reestablishing blood flow. A common percutaneous technique is referred to as balloon angioplasty where a balloon-tipped catheter is introduced to a blood vessel (e.g., typically through an introducing catheter). The balloon-tipped catheter is then advanced to the point of the occlusion and inflated to dilate the stenosis. Balloon angioplasty is appropriate for treating vessel stenosis, but it is generally not effective for treating acute thromboembolisms as none of the occlusive material is removed and the vessel will re-stenos after dilation. Another percutaneous technique involves placing a catheter near the clot and infusing streptokinase, urokinase, or other thrombolytic agents to dissolve the clot. Unfortunately, thrombolysis typically takes hours to days to be successful. Additionally, thrombolytic agents can cause hemorrhage and in many patients the agents cannot be used at all.

Various devices exist for performing a thrombectomy or removing other foreign material. However, such devices have been found to have structures which are either highly complex, cause trauma to the treatment vessel, or lack sufficient retaining structure and thus cannot be appropriately fixed against the vessel to perform adequately. Furthermore, many of the devices have highly complex structures that lead to manufacturing and quality control difficulties as well as delivery issues when passing through tortuous or small diameter catheters. Less complex devices may allow the user to pull through the clot, particularly with inexperienced users, and such devices may not completely capture and/or collect all the clot material.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment system including a catheter in accordance with embodiments of the present technology.

FIGS. 2A and 2B are an enlarged partial cut-away side and an isometric view, respectively, of a portion of the catheter of the system of FIG. 1 in accordance with embodiments of the present technology.

FIGS. 3A and 3B are an enlarged isometric view and a proximally-facing longitudinal view, respectively, of a distal portion of the catheter of FIG. 1 in accordance with embodiments of the present technology. FIG. 3C is an interior view of the catheter from within a lumen of the catheter in accordance with embodiments of the present technology. FIG. 3D is a partially-transparent isometric view of the catheter in accordance with embodiments of the present technology.

FIG. 4 is a partially-transparent isometric view of the catheter of FIG. 1 in accordance with additional embodiments of the present technology.

FIG. 5 is an interior view of the catheter of FIG. 1 from within a lumen of the catheter in accordance with additional embodiments of the present technology.

FIGS. 6A and 6B are an enlarged isometric view and an enlarged cross-sectional side view, respectively, of a distal portion of the catheter of FIG. 1 in accordance with additional embodiments of the present technology.

FIGS. 7A and 7B are side views of a distal portion of the clot treatment system of FIG. 1 during a procedure for removing clot material from within a blood vessel of a patient in accordance with embodiments of the present technology.

FIGS. 8A and 8B are a proximally-facing longitudinal view and an enlarged partially-transparent side view, respectively, of a distal portion of the catheter of FIG. 1 with clot material ingested therein in accordance with embodiments of the present technology.

FIGS. 9A and 9B are perspective views of the clot treatment system of FIG. 1 traversing a simulated pathway to the right pulmonary artery and the left pulmonary artery, respectively, in accordance with embodiments of the present technology. FIGS. 9C and 9D are distally-facing longitudinal views of a proximal portion of the catheter of FIG. 1 illustrating a flow pattern during aspiration without any interior grooves and with an arrangement of grooves shown in FIGS. 3A-3D, respectively, when the catheter traverses the simulated pathway to the right pulmonary artery shown in FIG. 9A in accordance with embodiments of the present technology.

FIG. 10A is a graph of a measured flow rate through the catheter of FIG. 1 versus a number of revolutions of interior grooves along the length of the catheter for the simulated pathway to the right pulmonary artery shown in FIG. 9A in accordance with embodiments of the present technology. FIG. 10B is a graph of a corresponding measured time to aspirate an occlusive synthetic clot through the catheter in accordance with embodiments of the present technology.

FIG. 11A is a graph of a measured distance traveled of an occlusive synthetic clot versus the number of revolutions of interior grooves along the length of the catheter when the clot was aspirated through the catheter of FIG. 1 for the simulated pathway to the right pulmonary artery shown in FIG. 9A in accordance with embodiments of the present technology. FIG. 11B is a graph of a corresponding measured clot velocity in accordance with embodiments of the present technology.

FIG. 12 is a graph of a measured maximum force required to move an occlusive synthetic clot through the catheter of FIG. 1 versus the number of revolutions of interior grooves along the length of the catheter when the clot was aspirated through the catheter for the simulated pathway to the right pulmonary artery shown in FIG. 9A in accordance with embodiments of the present technology.

FIG. 13 is a longitudinal view of a mandrel over which the catheter of FIG. 1 can be formed in accordance with embodiments of the present technology.

FIGS. 14A and 14B are a cross-sectional longitudinal view and an enlarged isometric view, respectively, of a mandrel over which the catheter of FIG. 1 can be formed in accordance with additional embodiments of the present technology.

### DETAILED DESCRIPTION

The present technology is generally directed to aspiration catheters having grooved inner surfaces for removing unwanted material from a patient, such as clot material from the vasculature of the patient, and associated systems and methods. In some embodiments, an aspiration catheter can include a proximal terminus, a distal terminus, and an inner surface defining a lumen. The inner surface includes at least one groove formed therein that extends at least partially between the distal terminus and the proximal terminus. When clot material is aspirated through the aspiration catheter, the at least one groove can provide a leak path past the clot material to improve the ingestion of the clot material through the catheter and inhibit clogging.

In some embodiments, the lumen extends about a longitudinal axis and the at least one groove can revolve circumferentially about the longitudinal axis between the proximal terminus and the distal terminus. Accordingly, the at least one groove can have a spiral/helical shape along the length of the aspiration catheter. In some aspects of the present technology, the spiral/helical shape of the at least one groove can generate a helical flow pattern within the lumen when clot material is aspirated through the aspiration catheter. The helical flow pattern can act to elongate and/or break apart the clot material and can increase the speed at which the clot material is ingested.

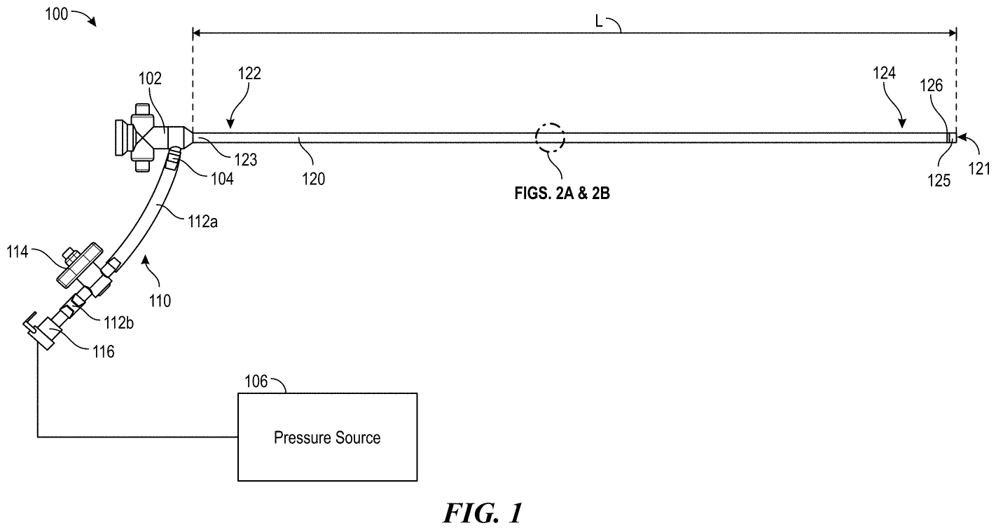

Certain details are set forth in the following description and in FIGS. 1-14B to provide a thorough understanding of various embodiments of the present technology. In other instances, well-known structures, materials, operations, and/or systems often associated with intravascular procedures, clot removal procedures, clot aspiration, catheters, and the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

The headings provided herein are for convenience only and should not be construed as limiting the subject matter disclosed.

### I. SELECTED EMBODIMENTS OF CLOT TREATMENT SYSTEMS

FIG. 1 is a partially schematic side view of a clot treatment system 100 in accordance with embodiments of the present technology. The clot treatment system 100 can also be referred to as an aspiration assembly, a clot removal system, a thrombectomy system, and/or the like. Although referred to as a clot treatment system and generally described in the context of removing clot material from the vasculature of the patient, the system 100 can be used to treat and/or remove other unwanted matter from the vasculature, body ducts, and/or other lumens of a patient. For example, the system 100 can be used to treat and/or remove emboli, foreign bodies, vegetation, and other materials from within the vasculature of a patient, gall stones from the gallbladder, and/or other materials from other body lumens.

In the illustrated embodiment, the clot treatment system 100 includes a tubing assembly 110 fluidly coupled to a catheter 120 via a valve 102. The catheter 120 can be referred to as an aspiration catheter, a guide catheter, an aspiration guide catheter, and/or the like. In general, the clot treatment system 100 (i) can include features generally similar or identical to those of the clot treatment systems described in detail in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

In the illustrated embodiment, the catheter 120 includes a proximal region or portion 122 and a distal region or portion 124 adjacent to and distal of the proximal portion 122. The catheter 120 further defines a lumen 121 extending entirely therethrough from the proximal portion 122 to the distal portion 124. The proximal portion 122 defines a proximal terminus 123 of the catheter 120, and the distal portion 124 defines a distal tip or terminus 125 of the catheter 120. In the illustrated embodiment, the distal portion 124 includes a marker band 126, such as a radiopaque marker configured to facilitate visualization of the position of the catheter 120 during a medical procedure (e.g., a clot removal procedure) using the catheter 120. In some embodiments, the catheter 120 can have a length L of between about 20-50 inches, between about 30-40 inches, about 35 inches, and so on.

The valve 102 is fluidly coupled to the lumen 121 of the catheter 120 and can be integral with or coupled to the proximal portion 122 of the catheter 120. In some embodiments, the valve 102 is a hemostasis valve that is configured to maintain hemostasis during a clot removal procedure by inhibiting or even preventing fluid flow in the proximal direction through the valve 102 as various components such as delivery sheaths, pull members, guidewires, interventional devices, other aspiration catheters, and the like are inserted through the valve 102 to be delivered through the catheter 120 to a treatment site in a blood vessel. The valve 102 includes a branch or side port 104 configured to fluidly couple the lumen 121 of the catheter 120 to the tubing assembly 110. In some embodiments, the valve 102 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety.

In the illustrated embodiment, the tubing assembly 110 fluidly couples the catheter 120 to a pressure source 106, such as a syringe (e.g., an auto-pressure locking syringe). The tubing assembly 110 can include (i) one or more tubing sections 112 (individually identified as a first tubing section 112a and a second tubing section 112b), (ii) at least one fluid control device 114 (e.g., a valve), and (iii) at least one connector 116 (e.g., a Toomey tip connector) for fluidly coupling the tubing assembly 110 to the pressure source 106 and/or other suitable components. In some embodiments, the fluid control device 114 is a stopcock that is fluidly coupled to (i) the side port 104 of the valve 102 via the first tubing section 112a and (ii) the connector 116 via the second tubing section 112b. The fluid control device 114 is externally operable by a user to regulate the flow of fluid therethrough and, specifically, from the lumen 121 of the catheter 120 to the pressure source 106. For example, the fluid control device 114 can be actuated to fluidly connect and fluidly disconnect the pressure source 106 from the lumen 121 of the catheter 120. In some embodiments, the connector 116 is a quick-release connector (e.g., a quick disconnect fitting) that enables rapid coupling/decoupling of the catheter 120 and the fluid control device 114 to/from the pressure source 106.

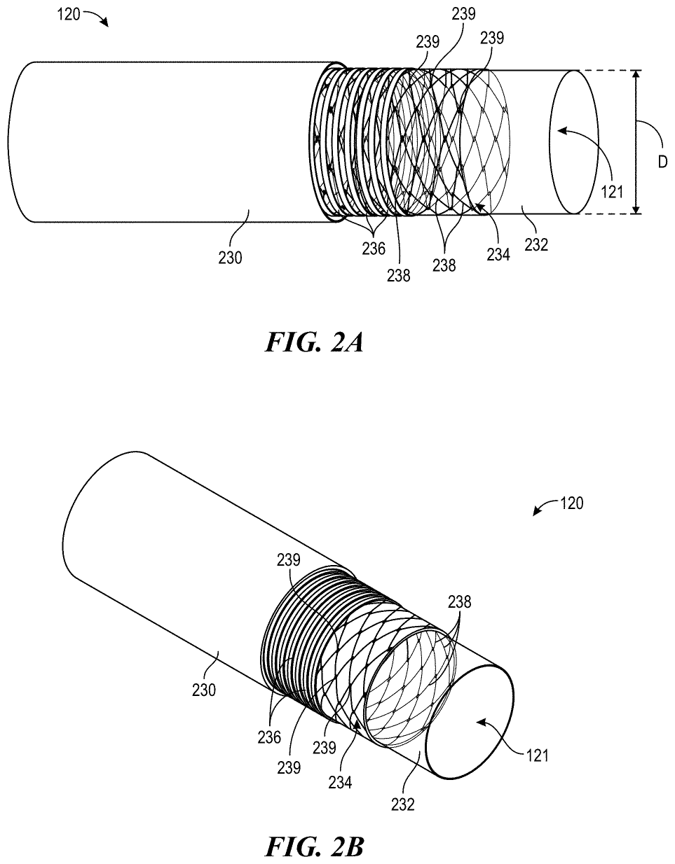

FIGS. 2A and 2B are an enlarged partial cut-away side and an isometric view, respectively, of a portion of the catheter 120 of FIG. 1 in accordance with embodiments of the present technology. Referring to FIGS. 2A and 2B together, the catheter 120 includes an outer sheath 230 and an inner liner 232. The outer sheath 230 is positioned over (e.g., radially outside of) the inner liner 232. The outer sheath 230 can also be referred to as an outer jacket, an outer shaft, or an outer layer, and the inner liner 232 can also be referred to as an inner layer, an inner sheath, or an inner shaft. In the illustrated embodiment, the catheter 120 further includes (i) a braid 234 extending between the outer sheath 230 and the inner liner 232 and (ii) a coil 236 extending between the outer sheath 230 and the braid 234. In some embodiments, the braid 234 and the coil 236 extend along the entire length L (FIG. 1) of the catheter 120. In some embodiments, the inner liner 232 can be omitted and the outer sheath 230 and/or another component of the catheter 120 can define an inner surface of the catheter 120.

In some embodiments, the outer sheath 230 is formed from a plastic material, elastomeric material, and/or thermoplastic elastomer (TPE) material. For example, the outer sheath 230 can be formed from a TPE manufactured by Arkema S.A., of Colombes, France, such as the TPEs manufactured under the trademark “Pebax.” The inner liner 232 defines the lumen 121 and can be formed of a lubricious material that facilitates the movement (e.g., distal advancement, proximal retraction) of various components through the lumen 121, such as clot material, delivery sheaths, pull members, guidewires, interventional devices, other aspiration catheters, and the like. In some embodiments, the inner liner 232 is formed from a polymer material, a fluoropolymer material (e.g., polytetrafluoroethylene (PTFE)), and/or another material having a high degree of lubricity. In some embodiments, the inner liner 232 has an inner diameter D (FIG. 2) of between about 0.2-0.5 inch (e.g., about 0.270 inch), greater than about 10 French, greater than about 16 French, greater than about 20 French, greater than about 24 French, or greater. In some embodiments, the diameter D is about 20 French or about 24 French.

The braid 234 can include wires, filaments, threads, sutures, fibers, or the like (collectively “wires 238”) that have been woven or otherwise coupled, attached, formed, and/or joined together at a plurality of interstices 239. Accordingly, the braid 234 can also be referred to as a braided structure, a braided filament structure, a braided filament mesh structure, a mesh structure, a mesh filament structure, and the like. The wires 238 can be formed from metals, polymers, and/or composite materials. In some embodiments, individual ones of the wires 238 are rolled flat wires having a cross-sectional dimension of between about 0.001-0.005 inch (e.g., about 0.002 inch) by about 0.002-0.005 inch (e.g., about 0.0033 inch).

The coil 236 can include a single wire wound around the braid 234 and the inner liner 232. In other embodiments, the coil 236 includes more than one wire wound about the braid 234 and/or the inner liner 232. For example, the coil 236 can include multiple wires wound over one another and/or multiple wires wound to at least partially overlap one another to form a braided or overlapping coil structure on the braid 234 and/or the inner liner 232. The coil 236 can be formed from a metallic or other suitably strong material, such as nickel-titanium alloys (e.g., nitinol), platinum, cobalt-chrome alloys, stainless steel, tungsten, and/or titanium.

In some embodiments, the construction of the catheter 120 can be selected/varied to provide a desired flexibility, strength, steerability, torque response, pushability, hoop strength, and/or other property. For example, in some embodiments the braid 234 and the coil 236 can extend through different regions of the catheter 120 (e.g., the proximal portion 122, the distal portion 124, an intermediate region therebetween, etc.) and/or only partially overlap. For example, the coil 236 can extend only through a distal region of the catheter 120 and can inhibit or even prevent kinking or other unwanted movement of the catheter 120 when the lumen 121 is aspirated during a clot removal procedure. Likewise, the hardness, thickness, and/or the like of the outer sheath 230 and the inner liner 232 can be varied in different regions of the catheter 120. For example, the outer sheath 230 and/or the inner liner 232 can be (i) relatively harder and/or thicker in the proximal portion 122 (FIG. 1) of the catheter 120 to provide the catheter 120 with good torque response, pushability, and/or steerability and (ii) relatively softer and/or thinner in the distal portion 124 (FIG. 1) such that the catheter 120 be steered to and positioned in difficult-to-reach regions of the anatomy (e.g., venous anatomy) of a patient while still having a relatively large size (e.g., 20 French, 24 French, greater than 24 French). In some embodiments, the catheter 120 can include some features that are at least generally similar in structure and function, or identical in structure and function, to those of the catheters disclosed in (i) U.S. patent application Publication Ser. No. 17/529,018, titled “CATHETERS HAVING SHAPED DISTAL PORTIONS, AND ASSOCIATED SYSTEMS AND METHODS,” and filed Nov. 17, 2021, and/or (ii) U.S. patent application Publication Ser. No. 17/529,064, titled “CATHETERS HAVING STEERABLE DISTAL PORTIONS, AND ASSOCIATED SYSTEMS AND METHODS,” and filed Nov. 17, 2021, each of which is incorporated herein by reference in its entirety.

### II. SELECTED EMBODIMENTS OF GROOVED ASPIRATION CATHETERS

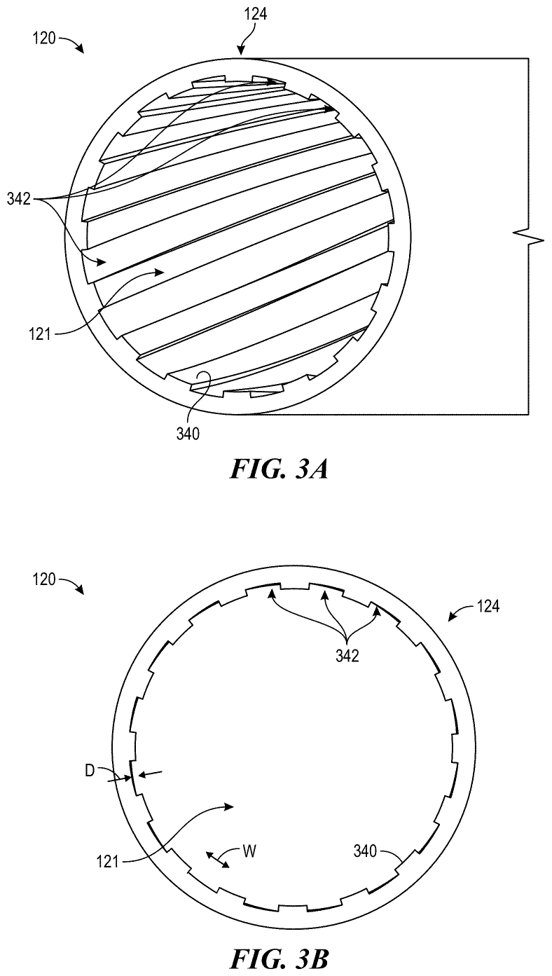

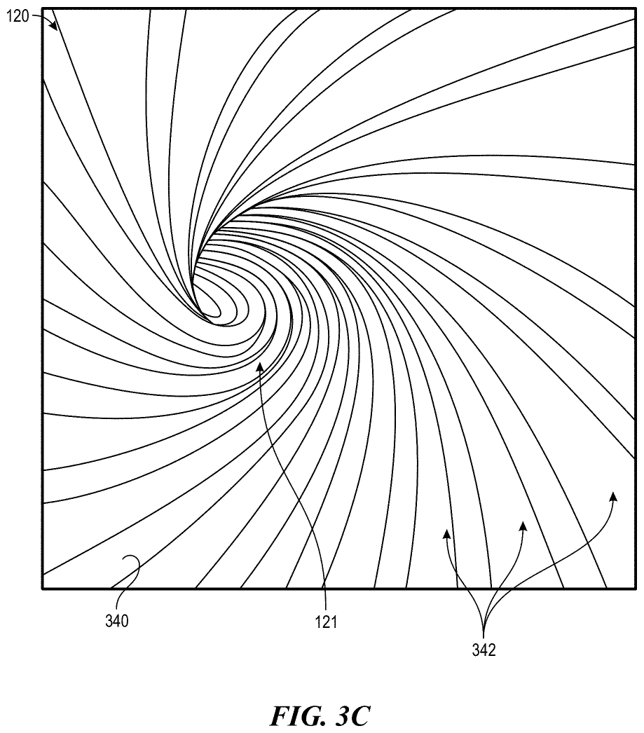

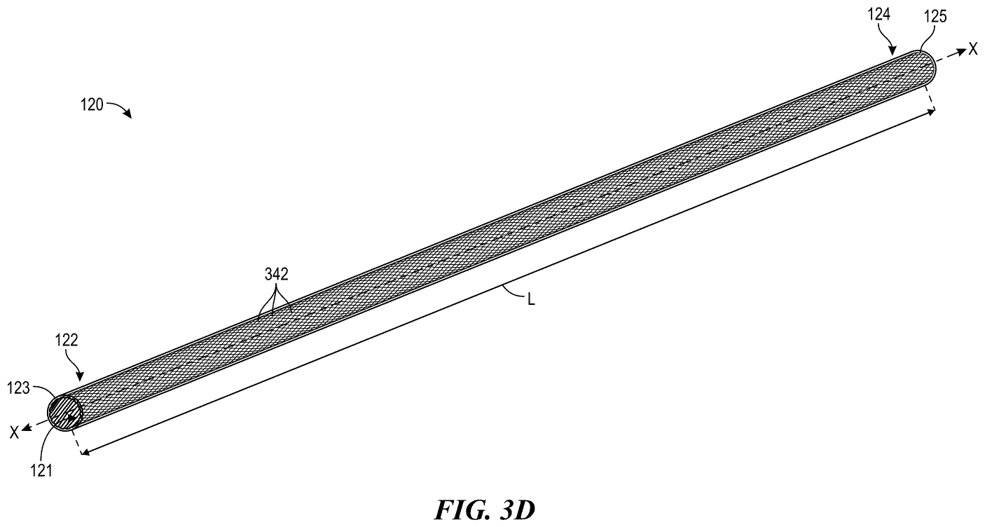

In some embodiments, the catheter 120 can include grooves on an inner surface thereof that are configured (e.g., sized and shaped) to improve the efficiency/effectiveness of clot aspiration with the catheter 120 during a clot treatment procedure. FIGS. 3A and 3B, for example, are an enlarged isometric view and a proximally-facing longitudinal view, respectively, of the distal portion 124 (e.g., the inlet) of the catheter 120 in accordance with embodiments of the present technology. FIG. 3C is an interior view of the catheter 120 from within the lumen 121 in accordance with embodiments of the present technology. And, FIG. 3D is a partially-transparent isometric view of the catheter 120 in accordance with embodiments of the present technology.

Referring to FIGS. 3A-3C together, the catheter 120 includes an inner surface 340 defining the lumen 121, and a plurality of grooves 342 (which can also be referred to as microchannels, microgrooves, channels, trenches, cuts, gutters, slits, and/or the like) formed along/in the inner surface 340. In some embodiments, the grooves 342 are formed in the inner liner 232 (FIGS. 2A and 2B). In the illustrated embodiment, the grooves 342 are identical and the catheter 120 includes sixteen of the grooves 342 equally spaced about a circumference of the inner surface 340—that is, equally spaced circumferentially about a longitudinal axis X of the catheter 120 (FIG. 3D) that extends through the lumen 121. The grooves 342 can each have a rectangular cross-sectional shape as shown in FIGS. 3A and 3B or the grooves 342 can each have a curved (e.g., semicircular) shape as shown in FIG. 3C. In other embodiments, the grooves 342 can have other cross-sectional shapes (e.g., rectilinear, polygonal, irregular) and/or different ones of the grooves 342 can have different cross-sectional shapes. In some embodiments, the grooves 342 have a thickness or depth D (FIG. 3B) of between about 0.003-0.100 inch (e.g., between about 0.005-0.050 inch, between about 0.005-0.0010 inch) and a width W (FIG. 3B) of between about 0.005-0.20 inch (e.g., between about 0.010-0.015 inch).

Referring to FIG. 3D, in the illustrated embodiment the grooves 342 each extend (i) along the entire length L of the catheter 120 between the proximal terminus 123 and the distal terminus 125 and (ii) extend circumferentially about the longitudinal axis X along the length L of the catheter 120. That is, the grooves 342 can revolve around the longitudinal axis L to form a spiral or helix pattern. In the illustrated embodiment, each of the grooves 342 traverses four complete revolutions about the longitudinal axis X along the length L of the catheter 120. Accordingly, in some aspects of the present technology the grooves 342 form a rifling pattern on the inner surface 340 (FIGS. 3A-3C) of the catheter 120. In some embodiments, the length L can be about 36 inches such that catheter 120 has about 0.14 revolutions per inch (e.g., 0.1380 revolutions per inch, between about 0.10-0.20 revolutions per inch, between about 0.13-0.16 revolutions per inch).

Referring to FIGS. 3A-3D together, in other embodiments the arrangement of the grooves 342 can be varied based on for example, a particular application of the catheter 120 (e.g., a particular clot removal procedure to be carried out with the catheter 120) and/or a desired aspiration flow pattern. For example, (i) the number of the grooves 342, (ii) the number of revolutions the grooves 342 traverse along the length L of the catheter 120, (iii) the cross-sectional shape of the grooves 342, (iv) the width W and/or depth D of the grooves 342, (v) the extent of the grooves 342 along the longitudinal axis X, and so on can be varied. In some embodiments, the catheter 120 can include between 1-20 or more of the grooves 342, and the grooves 342 can traverse between 0-20 revolutions along the length L of the catheter 120.

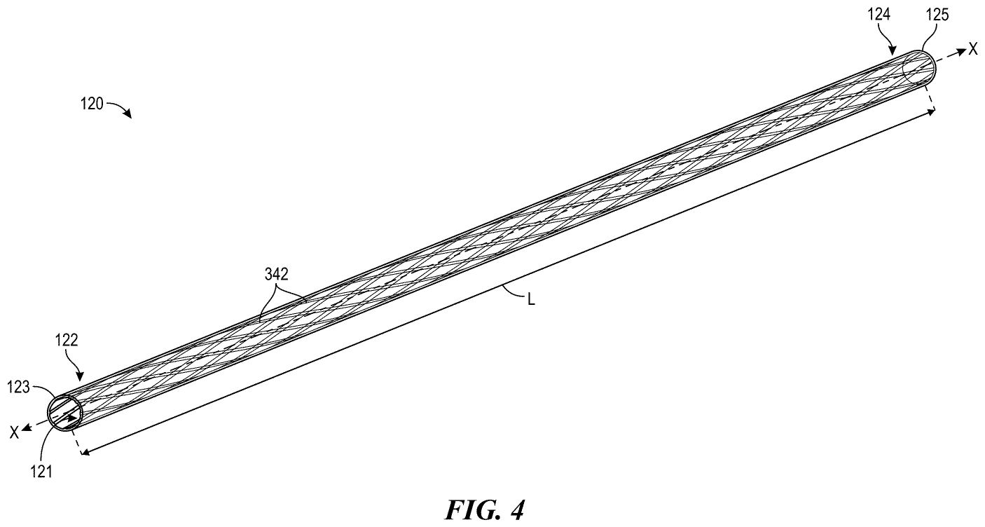

For example, FIG. 4 is a partially-transparent isometric view of the catheter 120 in accordance with additional embodiments of the present technology. In the illustrated embodiment, the catheter 120 includes eight of the grooves 342 equally spaced circumferentially about the longitudinal axis X and extending along the entire length L of the catheter 120 between the proximal terminus 123 and the distal terminus 125. Further, the grooves 342 each traverse two full revolutions about the longitudinal axis X along the length L of the catheter 120. In some embodiments, the length L can be about 36 inches such that catheter 120 has about 0.053 revolutions per inch (e.g., 0.0526 revolutions per inch, between about 0.02-0.09 revolutions per inch, between about 0.04-0.07 revolutions per inch).

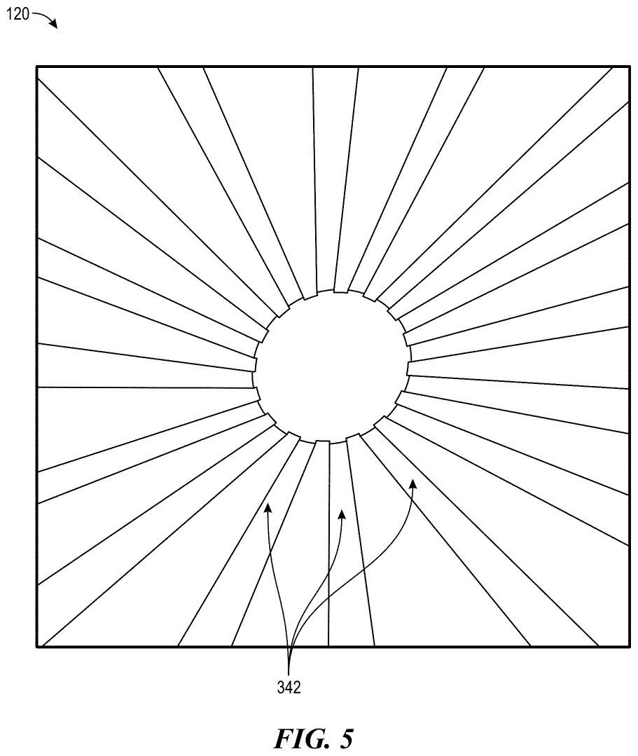

For example, FIG. 5 is an interior view of the catheter 120 from within the lumen 121 in accordance with additional embodiments of the present technology. In the illustrated embodiment, the catheter 120 includes sixteen of the grooves 342 and the grooves 342 extend generally parallel to the longitudinal axis X (FIG. 3D; extending into the page in FIG. 5). That is, the grooves 342 do not revolve circumferentially around the axis X along the length of the catheter 120 (e.g., the grooves 342 traverse zero revolutions per inch).

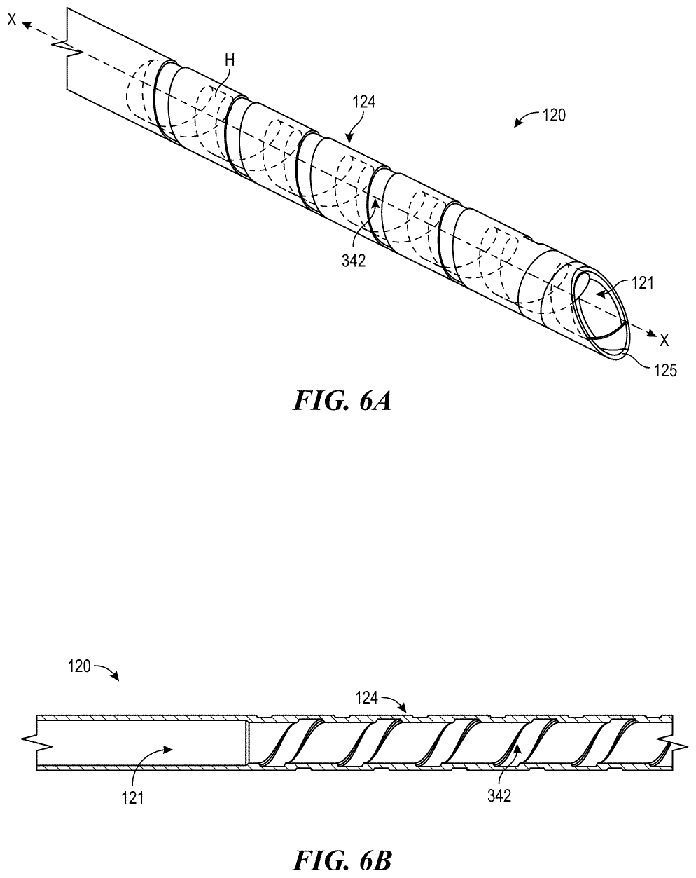

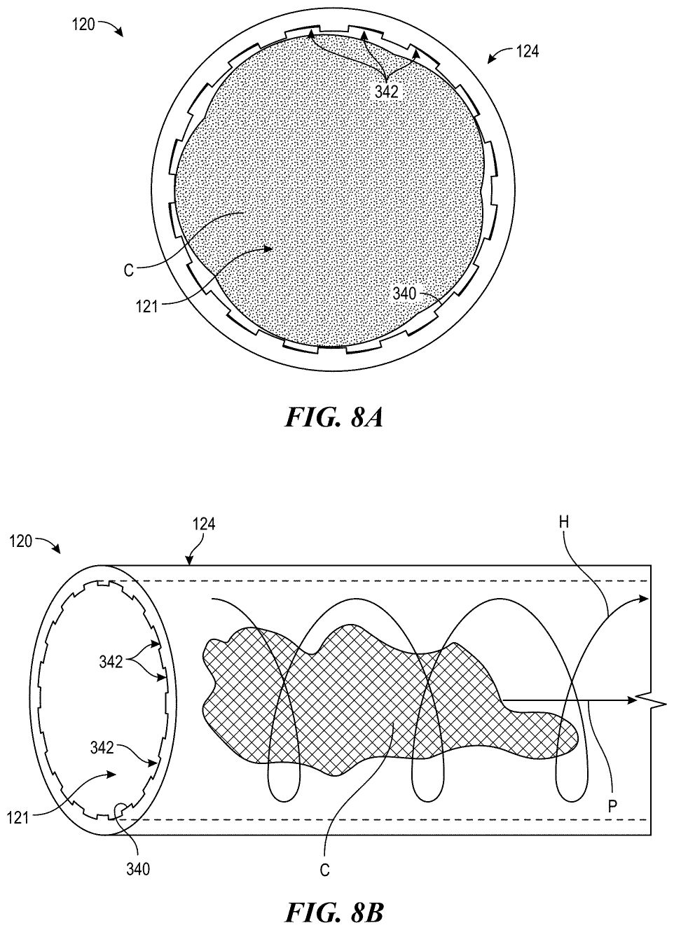

For example, FIGS. 6A and 6B are an enlarged isometric view and an enlarged cross-sectional side view, respectively, of the distal portion 124 of the catheter 120 in accordance with additional embodiments of the present technology. Referring to FIGS. 6A and 6B together, in the illustrated embodiment the catheter 120 includes a single one of the grooves 342 that extends from the distal terminus 125 only partially toward the proximal terminus 123 (FIG. 1) of the catheter 120. That is, the groove 342 does not extend along the full length L (FIG. 1) of the catheter 120 but only partially through the distal portion 124. Moreover, in the illustrated embodiment the groove 342 extends circumferentially (e.g., spirals) about the longitudinal axis X (FIG. 6A) six times before terminating at a proximal end portion. In other embodiments, the groove 342 can revolve more or fewer times about the longitudinal axis X. In some embodiments, the depth D (FIG. 3B) of the groove 342 is between about 0.010-0.015 inch and the width W (FIG. 3B) is between about 0.120-0.145 inch. As described in greater detail below with reference to FIG. 8B, the groove 342 can be configured (e.g., shaped and/or sized) to generate a helical flow pattern within the lumen 121 when the catheter 120 is aspirated as indicated by arrow H (which spirals/revolves about the lumen 121) in FIG. 6A.

Referring to FIGS. 3A-6B together, in other embodiments the grooves 342 need not extend from the distal terminus of 124 of the catheter 120 and can instead start along a middle portion of the catheter 120 between the proximal terminus 123 and the distal terminus 125. That is, the grooves 342 can be spaced apart from the distal terminus 125 and need not extend along the full length L (FIG. 1) of the catheter 120. Moreover, the grooves 342 can be equally spaced relative to one another or can be spaced at varying distances relative to one another.

### III. SELECTED EMBODIMENTS OF CLOT TREATMENT METHODS

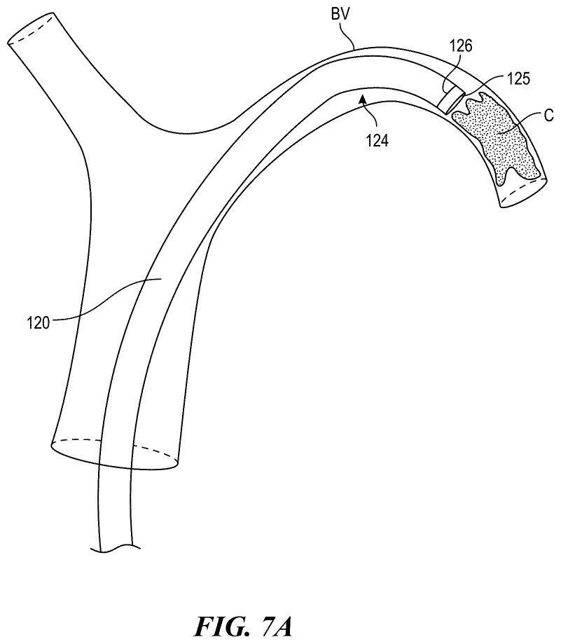

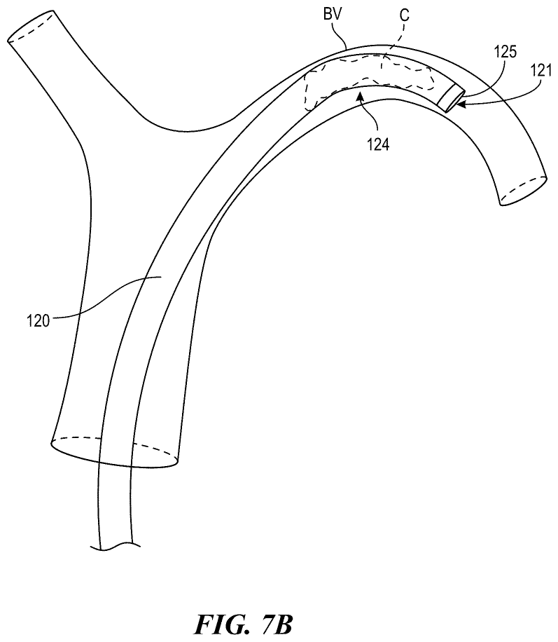

FIGS. 7A and 7B are side views of the distal portion 124 of the catheter 120 of the clot treatment system 100 of FIG. 1 during a procedure for removing clot material C (e.g., a pulmonary embolism) from within a blood vessel BV (e.g., a pulmonary blood vessel) of a patient (e.g., a human patient) in accordance with embodiments of the present technology. As noted above, in some embodiments the clot removal procedure illustrated in FIGS. 7A and 7B can be generally similar or identical to any of the clot removal procedures disclosed in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety.

With reference to FIGS. 1 and 7A together, the catheter 120 can be advanced through the patient to proximate the clot material C with the blood vessel BV (e.g., advanced to a treatment site within the blood vessel BV). In some embodiments, the catheter 120 can be advanced through the blood vessel BV until the distal terminus 125 of the catheter 120 is positioned proximate to a proximal portion of the clot material C. In some embodiments, the position of the distal terminus 125 can be confirmed or located via visualization of the marker band 126 using fluoroscopy or another imaging procedure (e.g., a radiographic procedure). In other embodiments, the distal terminus 125 can be positioned at least partially within the clot material C or distal of the clot material C.

Access to the pulmonary vessels can be achieved through the patient's vasculature, for example, via the femoral vein. In some embodiments, the clot treatment system 100 can include an introducer (e.g., a Y-connector with a hemostasis valve; not shown) that can be partially inserted into the femoral vein. A guidewire (not shown) can be guided into the femoral vein through the introducer and navigated through the right atrium, the tricuspid valve, the right ventricle, the pulmonary valve, and into the main pulmonary artery. Depending on the location of the clot material C, the guidewire can be guided to one or more of the branches of the right pulmonary artery and/or the left pulmonary artery. In some embodiments, the guidewire can be extended entirely or partially through the clot material C. In other embodiments, the guidewire can be extended to a location just proximal of the clot material C. After positioning the guidewire, a dilator and the catheter 120 can be placed over the guidewire and advanced to the position proximate to the clot material C as illustrated in FIG. 7A. The dilator can then be withdrawn proximally through the lumen 121 of the catheter 120. In some embodiments, the guidewire can then be withdrawn while, in other embodiments, the guidewire can remain and can be used to guide other catheters (e.g., delivery catheters, additional aspiration guide catheters), interventional devices, and the like to the treatment site. It will be understood, however, that other access locations into the venous circulatory system of a patient are possible and consistent with the present technology. For example, the user can gain access through the jugular vein, the subclavian vein, the brachial vein, or any other vein that connects or eventually leads to the superior vena cava. Use of other vessels that are closer to the right atrium of the patient's heart can also be advantageous as it reduces the length of the instruments needed to reach the clot material C.

With reference to FIGS. 1 and 7B together, the pressure source 106 is configured to generate (e.g., form, create, charge, build-up) a vacuum (e.g., negative relative pressure) and store the vacuum for subsequent application to the catheter 120. For example, after positioning the catheter 120 proximate the clot material C, a user can first close the fluid control device 114 before generating the vacuum in the pressure source 106 by, for example, withdrawing the plunger of a syringe coupled to the connector 116. In this manner, a vacuum is charged within the pressure source 106 (e.g., a negative pressure is maintained) before the pressure source 106 is fluidly connected to the lumen 121 of the catheter 120. To aspirate the lumen 121 of the catheter 120, the user can open the fluid control device 114 to fluidly connect the pressure source 106 to the catheter 120 and thereby apply or release the vacuum stored in the pressure source 106 to the lumen 121 of the catheter 120.

Opening of the fluid control device 114 instantaneously or nearly instantaneously applies the stored vacuum pressure to the tubing assembly 110 and the catheter 120, thereby generating a suction pulse throughout the catheter 120. In particular, the suction is applied at the distal portion 124 of the catheter 120 to suck/aspirate/ingest at least a portion of the clot material C into the lumen 121 of the catheter 120, as shown in FIG. 7B. In one aspect of the present technology, pre-charging or storing the vacuum in the pressure source 106 before applying the vacuum to the lumen 121 of the catheter 120 is expected to generate greater suction forces and corresponding fluid flow velocities at and/or near the distal terminus 125 of the catheter 120 compared to simply activating the pressure source 106 while it is fluidly connected to the catheter 120. The vacuum pressure can act to draw the clot material C down the entire length of the catheter 120, through the side port 104, through the tubing assembly 110, and into the pressure source 106.

Sometimes, as shown in FIG. 7B, discharging the vacuum stored in the pressure source to aspirate the lumen 121 of the catheter 120 may remove substantially all (e.g., a desired amount) of the clot material C from the blood vessel BV. That is, a single aspiration pulse may adequately remove the clot material C from the blood vessel BV. In other embodiments, a portion of the clot material C may remain in the blood vessel BV. In such instances, the user may wish to again apply vacuum pressure (conduct another “aspiration pass”) to remove all or a portion of the remaining clot material C in the blood vessel BV. In such instances, the pressure source 106 can be disconnected from the tubing assembly 110 and drained (e.g., aspirated clot removal removed) before the pressure source 106 is reconnected to the tubing assembly 110 and activated once again. After removing a desired amount of the clot material C, the catheter 120 can be withdrawn from the patient.

Sometimes, however, the clot material C can clog and become stuck within the lumen 121 of the catheter 120 and/or around the distal terminus 125 of the catheter 120 (e.g., forming a “lollipop” around the distal terminus 125). Clearing such clogs can require (i) performing additional aspiration passes, (ii) removing the entire catheter 120 from the patient and then reinserting the same or a different catheter 120 for another aspiration pass, (iii) and/or inserting an additional clot removal element through the catheter 120 to mechanically disrupt and dislodge the clog. Such techniques to clear the clog can increase the complexity and time of the clot removal procedure.

In some aspects of the present technology, the grooves 342 (FIGS. 3A-6B) of the catheter 120 can facilitate improved ingestion/aspiration of the clot material C into and through the catheter 120—thereby inhibiting clogging—as compared to, for example, conventional catheters having a non-grooved inner surface. FIGS. 8A and 8B, for example, are a proximally-facing longitudinal view and an enlarged partially-transparent side view, respectively, of the distal portion 124 (e.g., inlet) of the catheter 120 with the clot material C positioned therein in accordance with embodiments of the present technology. In the illustrated embodiment, the catheter 120 includes the arrangement of the grooves 342 shown in FIGS. 3A-3D.

Referring to FIG. 8A, some or all of the grooves 342 can create fluid paths (e.g., leak paths, microfluid paths, microleak paths) around the clot material C. These fluid paths can inhibit or prevent the clot material C from clogging the lumen 121 and causing a cavitation within the pressure source 106 by keeping blood flowing generally proximally through the catheter 120 through the grooves 342 during aspiration. This movement of the blood through the grooves 342 can help pull the clot material C farther proximally into and through the lumen 121 of the catheter 120. Similarly, the grooves 342 can also reduce the area of the inner surface 340 that contacts the clot material C—thereby reducing friction between the clot material C and the catheter 120, and ultimately the aspiration force required to move the clot material C proximally through the entire length of the catheter 120.

Referring to FIG. 8B, the rifling pattern on the grooves 342 (e.g., revolving circumferentially about the longitudinal axis of the catheter 120) can create a helical flow pattern inside the lumen 121 of the catheter 120 during aspiration as indicated by the arrow H. In some aspects of the present technology, the helical flow pattern can help keep faster moving blood travelling proximally through the lumen 121 near the inner surface 340 while maintaining the slower moving clot material C toward the center of the lumen 121. The outer “layer” of blood near the inner surface 340 can create a fluid buffer between the clot material C and the inner surface 340 of the catheter 120 that facilitates a more efficient and reliable transport of the clot material C down the entire length of the catheter 120 and into the pressure source 106 (FIG. 1). More specifically, the helical flow of the blood can (i) exert an axial force against the clot material C that pulls the clot material C in a proximal direction indicated by arrow P via suction on the clot and (ii) exert a torsional force against the clot material C as indicated by the arrow H. These forces can apply shear and torsional stresses against the clot material C that act to deform (e.g., lengthen) and/or break apart the clot material C within the lumen 121 in a manner in which laminar and/or non-helical flow would not. This can further reduce the area of the inner surface 340 that contacts the clot material C— thereby reducing friction between the clot material C and the catheter 120. The decrease in friction and/or deformation of the clot material C can help the clot material C travel through the lumen 121 without clogging the lumen 121.

### IV. SELECTED EXAMPLES OF THE PERFORMANCE OF EMBODIMENTS OF THE PRESENT TECHNOLOGY AND ASSOCIATED OPTIMIZATIONS

Referring to FIGS. 3A-6B together, as described above, the arrangement of the grooves 342 can be varied to provide different aspiration flow patterns, flow rates, and/or the like. In general, for example, it is expected that increasing the number of the grooves 342 will improve/strengthen the axial flow path past an ingested clot up to a point at which the grooves 342 are too small to provide an efficient leak path past the ingested clot material. Likewise, it is expected that increasing the depth D of the grooves 342 will improve the helical flow pattern at the cost of increasing the wall thickness of the catheter 120 (e.g., the thickness of the inner liner 232 shown in FIGS. 2A and 2B) that the grooves 342 are formed in. For example, significant increases in the depth D are expected to require a corresponding increase in the overall thickness of the inner liner 232 (e.g., an increase in the thickness of the inner liner 232 radially outside the grooves 342—i.e., on the outer side or back side of the grooves 342) to maintain the integrity of the inner liner 232. Increasing the wall thickness of the catheter 120 can decrease the flexibility of the catheter 120. Therefore, for example, the grooves 342 can be relatively deeper for applications in which the catheter 120 does not need to be made very flexible (e.g., for clot removal procedures in non-tortuous anatomies), while the grooves 342 can be made shallower for applications in which it is advantageous for the catheter 120 to be made very flexible (e.g., for clot removal procedures in tortuous anatomies such as the pulmonary arteries).

Similarly, it is expected that increasing the number of revolutions of the grooves 342 (where the grooves 342 are rifled along the length L of the catheter 120) will (i) improve the helical flow pattern within the catheter 120 thereby increasing the torsional forces acting to lengthen and break apart the ingested clot material while also (ii) decreasing the volumetric flow rate within the catheter 120 by increasing the length the blood must travel through the catheter 120. However, where the catheter 120 traverses a tortuous path, the linear/laminar flow provided by a catheter without grooves or with linear (e.g., non-revolving) grooves can impede flow as the flow must change its trajectory down the length of the catheter. In some aspects of the present technology, the rifled arrangement of the grooves 342 can improve the flow pattern and/or volumetric flow rate of the catheter 120 in tortuous anatomies as the blood flow need not change its trajectory through the tortuous path of the catheter 120 as much.

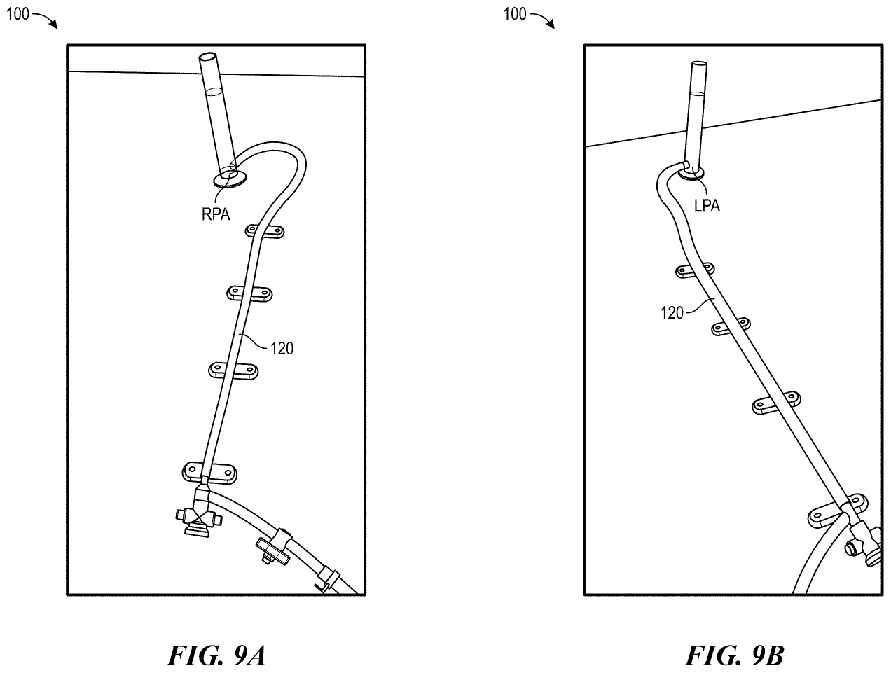

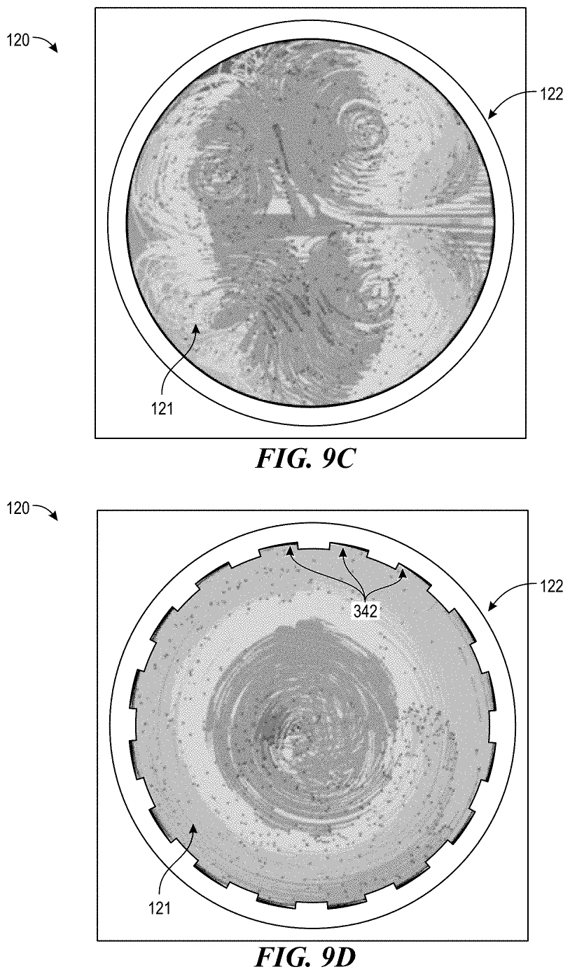

More specifically, for example, FIGS. 9A and 9B are perspective views of the clot treatment system 100 traversing a simulated pathway to a right pulmonary artery RPA and a left pulmonary artery LPA, respectively, in accordance with embodiments of the present technology. Referring to FIGS. 9A and 9B together, the pathway to the right pulmonary artery RPA can be more tortuous than the pathway to left pulmonary artery LPA. More specifically, in the illustrated embodiment the pathway to the right pulmonary artery RPA has a tortuosity defined as the amount of curvature of the catheter 120 divided by the length L (FIG. 1) of the catheter 120 of about 1.40, while the pathway to the left pulmonary artery LPA has a tortuosity of about 1.06.

FIGS. 9C and 9D are distally-facing longitudinal views of the proximal portion 122 (e.g., the outlet) of the catheter 120 illustrating a flow pattern during aspiration without any of the grooves 342 and with the arrangement of the grooves 342 shown in FIGS. 3A-3D, respectively, when the catheter 120 traverses the simulated pathway to the right pulmonary artery RPA shown in FIG. 9A in accordance with embodiments of the present technology. Referring to FIGS. 9C and 9D together, in both embodiments the fastest flow is toward the center of the lumen 121. However, the flow pattern generated by the catheter 120 without the grooves 342 is much less uniform than the flow pattern generated by the catheter 120 with the grooves 342. In some aspects of the present technology, the nonuniform flow pattern shown in FIG. 9C traverses the lumen 121 of the catheter more slowly than the more uniform flow pattern shown in FIG. 9D—reducing the volumetric flow rate of the catheter 120 and the efficiency at which the catheter 120 can ingest clot material.

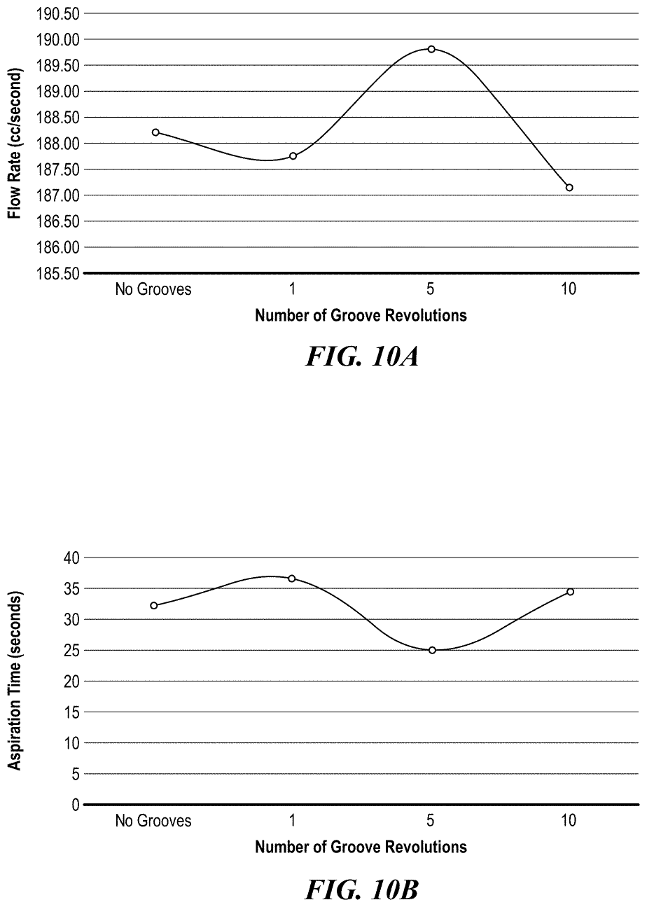

In some aspects of the present technology, the number of rotations that the grooves 342 traverse down the length L of the catheter 120 (and/or the number of rotations per unit length) can impact the flow rate and efficiency of clot aspiration through the catheter 120. For example, FIG. 10A is a graph of a measured flow rate through the catheter 120 versus the number of revolutions of the grooves 342 along the length of the catheter 120 for the simulated pathway to the right pulmonary artery shown in FIG. 9A in accordance with embodiments of the present technology. The graph illustrates the plot for the average flow rate when the catheter 120 had a size of 24 French and a length of about 36 inches. FIG. 10B is a graph of a corresponding measured time to aspirate an occlusive synthetic clot through the catheter 120 in accordance with embodiments of the present technology. The graph in FIG. 10B illustrates the plot for a synthetic clot that was formed of silicone 27A mold and had a spherical shape with diameter greater than the diameter of the catheter 120 of 0.28 inch. Referring to FIGS. 10A and 10B together, the flow rate was maximized, and the corresponding aspiration time was minimized, when the catheter 120 was tested with five revolutions (e.g., about 0.14 revolutions per inch) as opposed to having no grooves (e.g., 0 revolutions per inch), or grooves with one or ten revolutions (e.g., about 0.03 or 0.28 revolutions per inch).

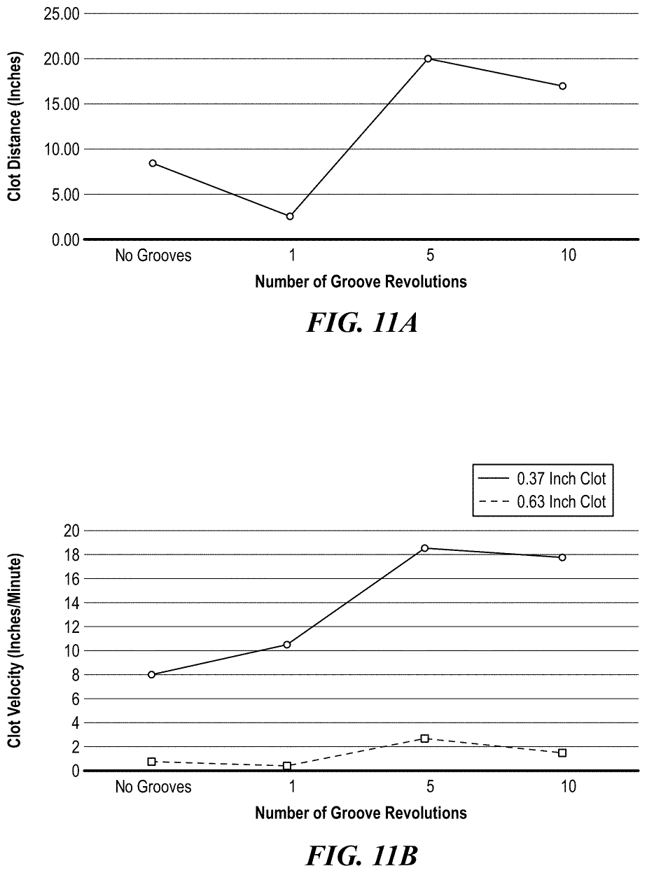

Similarly, FIG. 11A is a graph of a measured distance traveled of an occlusive synthetic clot through the catheter 120 versus the number of revolutions of the grooves 342 along the length of the catheter 120 when the clot was aspirated through the catheter 120 for the simulated pathway to the right pulmonary artery shown in FIG. 9A in accordance with embodiments of the present technology. The graph illustrates the plot for the average clot distance when the catheter 120 had a size of 24 French and a length of about 36 inches, and when the synthetic clot was formed of silicone 27A mold and had a cylindrical shape with a length of 0.63 inch and a diameter of 0.28 inch. FIG. 11B is a graph of a corresponding measured clot velocity in accordance with embodiments of the present technology. FIG. 11B further illustrates a plot for the average clot velocity for a synthetic clot having a cylindrical shape with a smaller length of 0.37 inch. Referring to FIGS. 11A and 11B together, again the distance the clot traveled and the velocity of the clot during aspiration was maximized when the catheter 120 was tested with five revolutions as opposed to having no grooves, or grooves with one or ten revolutions.

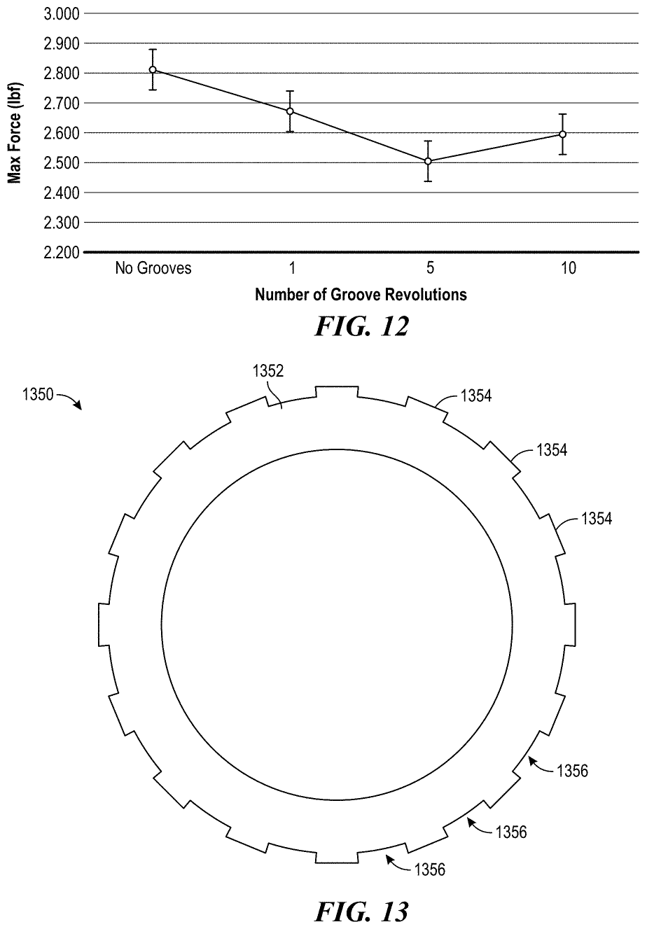

Similarly, FIG. 12 is a graph of a measured maximum force required to move an occlusive synthetic clot through the catheter 120 versus the number of revolutions of the grooves 342 along the length of the catheter 120 when the clot was aspirated through the catheter 120 for the simulated pathway to the right pulmonary artery shown in FIG. 9A in accordance with embodiments of the present technology. The graph illustrates the plot for the maximum force when the catheter 120 had a size of 24 French and a length of about 36 inches, and when the synthetic clot was formed of silicone 27A mold and had a cylindrical shape with a length of 0.50 inch. As shown, the maximum force was again minimized when the catheter 120 was tested with five revolutions as opposed to having no grooves, or grooves with one or ten revolutions. That is, five revolutions of the grooves 342 minimized the friction between the clot and the catheter 120 during aspiration.

Accordingly, in some aspects of the present technology five revolutions of the grooves 342—or about five revolutions—can maximize the efficiency of clot removal via aspiration through the catheter 120 by (i) increasing the flow rate through the catheter 120, (ii) reducing clot aspiration time through the catheter 120, (iii) increasing the distance the clot travels through the catheter 120, (iv) increasing the velocity at which the clot travels through the catheter 120, (v) decreasing the friction between the clot and the catheter 120, and/or (vi) decreasing the force needed to pull the clot through the catheter 120. It is expected that the optimum number of revolutions (among other parameters) can vary depending on the path traversed by the catheter 120 through a patient.

Moreover, in some aspects of the present technology it is expected that the optimum number of revolutions is dependent on the length of the catheter 120. Accordingly, the optimum number of revolutions per unit length of the catheter 120 can remain constant for catheters of different lengths. As set forth above, for example, the catheter 120 can include between about 0.01-0.40 revolutions per inch (e.g., about 0.028 revolutions per inch, about 0.056 revolutions per inch, about 0.139 revolutions per inch, about 0.278 revolutions per inch, between about 0.10-0.20 revolutions per inch, between about 0.12-0.16 revolutions per inch, etc.). That is, the catheter 120 can include between about 0.005-0.15 revolutions per centimeter (e.g., about 0.011 revolutions per centimeter, about 0.022 revolutions per centimeter, about 0.055 revolutions per centimeter, about 0.109 revolutions per centimeter, between about 0.03-0.08 revolutions per centimeter, between about 0.04-0.07 revolutions per centimeter, etc.).

### V. SELECTED EMBODIMENTS OF DEVICES, SYSTEMS, AND METHODS FOR MANUFACTURING GROOVED ASPIRATION CATHETERS

Referring to FIGS. 1-6B together, in some embodiments the catheter 120 can be formed about a mandrel, hypotube, or other elongate member. For example, the inner liner 232 can first be positioned about the mandrel and, in some embodiments, stretched along the mandrel to a desired thickness. Then, the braid 234 can be formed (e.g., wound, braided) about the inner liner 232 around the mandrel. Next, the coil 236 can be wound around the mandrel about the braid 234 and the inner liner 232. In some embodiments, the marker band 126 can be positioned about the mandrel in the distal portion 124. Next, the outer sheath 230 can be positioned over the inner liner 232, the braid 234, and the coil 236, and then some or all of these components can be heat shrunk, fused, laminated, or otherwise secured together.

To form the grooves 342, the mandrel can be formed with corresponding features that shape the inner liner 232. FIG. 13, for example, is a longitudinal view of a mandrel 1350 over which the catheter 120 can be formed in accordance with embodiments of the present technology. In the illustrated embodiment, the mandrel 1350 includes a body 1352 (e.g., a cylindrical body) and positive features 1354 (e.g., extrusions, ridges, projections) projecting radially outward from the body 1352 and separated by grooves or trenches 1356. Referring to FIGS. 1-6B and 13 together, the positive features 1354 can correspond to the desired pattern of the grooves 342. For example, the features 1354 can have dimensions corresponding to the desired depth D and width W of the grooves 342, and can spiral about the body 1352 any number of revolutions to provide the desired rifling pattern of the grooves 342. The number of the features 1354, the spacing between the features 1354, the size (e.g., height, width) of the features 1354, the pitch or helical pattern of the features 1354, and/or other parameters of the features 1354 can be selected to produce a pattern of the grooves 342 having a desired number, depth D, and width W. For example, the features 1354 have a rectangular cross-sectional shape in FIG. 13 to produce grooves 342 having a rectangular cross-sectional shape while, in other embodiments, the features 1354 can have other cross-sectional shapes (e.g., circular, polygonal, irregular, etc.) to produce grooves 342 of corresponding shape. As a further example, in FIG. 13 the mandrel 1350 includes 16 of the features 1354 such that the catheter 120 has a corresponding 16 of the grooves 342. The mandrel 1350 can have any number of the features 1354.

When the catheter 120 is formed (e.g., laminated) over the mandrel 1350, the catheter 120 can shrink radially about the mandrel 1350 and against/between the features 1354 to from the grooves 342. Specifically, the inner liner 232 can melt and flow into the trenches 1356 between the features 1354 before solidifying to form the grooves 342 (e.g., when the inner liner 232 comprises a Pebax material). Alternatively or additionally, the inner liner 232 can be pressed and/or formed into the trenches 1356 without melting (e.g., when the inner liner 232 comprises a PTFE material). In some embodiments, to facilitate removal of the catheter 120 from the mandrel 1350 after manufacturing, a lubricant (e.g., a silicone spray lubricant) can be applied to the mandrel 1350 prior to manufacturing of the catheter 120 (e.g., in a mold release process). Similarly, the mandrel 1350 can include a more permanent thin layer of PTFE coating over the outer surface thereof to facilitate removal and release of the catheter 120. In some embodiments, the manufacturing process can include a destructive process step that stretches and necks down the mandrel 1350 to a smaller outer diameter to facilitate removal and release of the catheter 120.

In some embodiments, the features 1354 can extend linearly along the length of the body 1352, and the mandrel 1350 can be fixed at one end and then rotated to provide the rifling pattern of the grooves 342. For example, the mandrel 1350 can be rotated a desired number of times during manufacturing (e.g., with the inner liner 232 in a molten state) such that the grooves 342 traverse a corresponding number of revolutions along the length L of the catheter 120. In some embodiments, the features 1354 can revolve at least partially about the body 1352, and the mandrel 1350 can be rotated during manufacturing of the catheter 120 to introduce further revolutions into the grooves 342.

The features 1354 of the mandrel 1350 can be formed by machining the body 1352 (e.g., a hyptotube or solid tube) to cut-out or etch the trenches 1356. In some embodiments, the machining is performed by a multi-axis machine that can rotate the mandrel 1350 during machining such that the trenches 1356 (and the corresponding features 1354) revolve about the body 1352. In other embodiments, the mandrel 1350 can be formed by extruding the body 1352 to form the positive features 1354 and the trenches 1356.

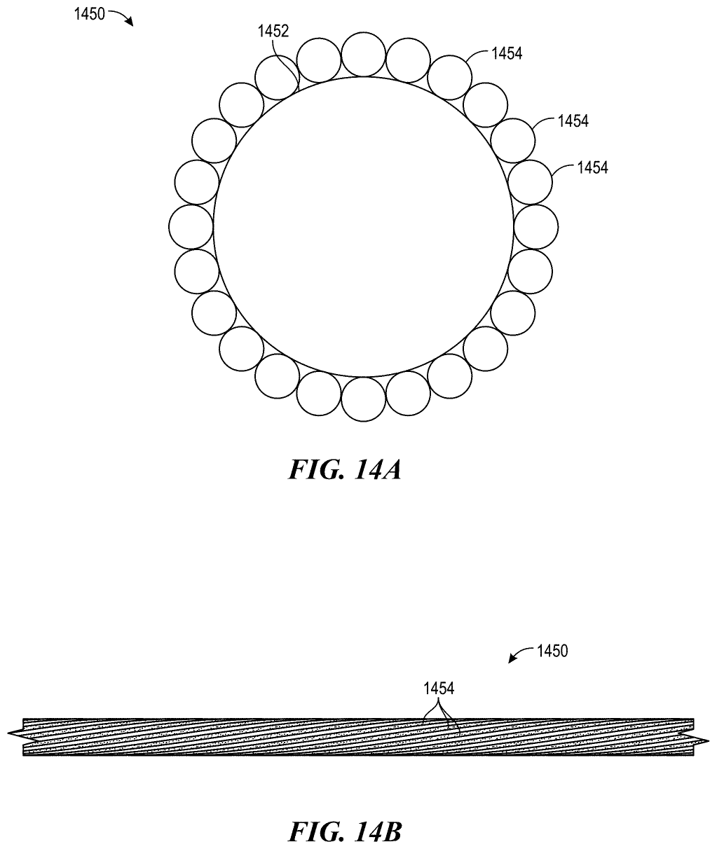

FIGS. 14A and 14B are a cross-sectional longitudinal view and an enlarged isometric view, respectively, of a mandrel 1450 over which the catheter 120 can be formed in accordance with additional embodiments of the present technology. Referring to FIGS. 14A and 14B together, in the illustrated embodiment the mandrel 1450 includes a body 1452 and filaments or wires 1454 (e.g., positive features) positioned about the body 1452 that correspond to the desired pattern of the grooves 342 (FIGS. 1-6B). The wires 1454 can be welded to the body 1452, tightly wound about the body 1452 (e.g., and secured at both ends of the wires 1454), or otherwise secured about/to the body 1452. Referring to FIGS. 1-6B, 14A, and 14B together, when the catheter 120 is formed about the mandrel 1450, the inner liner 232 can melt and flow into the spaces between the wires 1454 before solidifying to form the grooves 342, and/or can be pressed/formed into the spaces between the wires 1454 to form the grooves 342. The number of the wires 1454, the spacing between the wires 1454, the size (e.g., diameter) of the wires 1454, the pitch or helical pattern of the wires 1454, and/or other parameters of the wires 1454 can be selected to produce a pattern of the grooves 342 having a desired number, depth D, and width W. For example, in the illustrated embodiment the wires 1454 are evenly spaced circumferentially about the body 1452 and in contact with each other to form a more uniform pattern of the grooves 1454. Likewise, in FIGS. 14A and 14B, the mandrel 1450 includes 24 of the wires 1454 such that the catheter 120 has a corresponding 24 of the grooves 342. For example, reducing the number of the wires 1454 will produce a pattern having fewer of the grooves 342, and spacing the wires 1454 apart from one another will produce a pattern having a greater depth D and width W of the grooves. Similar to the positive features 1354 of FIG. 13, the wires 1454 can wrap helically about the body 1452 to correspond to the desired number of revolutions of the grooves 342, or the wires 1454 can extend linearly (or at least partially helically) along the body 1452 and the mandrel 1450 can be rotated during manufacturing to produce a revolving (e.g., helical) pattern of the grooves 342.

### VI. ADDITIONAL EXAMPLES

Several aspects of the present technology are set forth in the following examples:

1. An aspiration catheter, comprising:
a proximal terminus;
a distal terminus; and
an inner surface defining a lumen, wherein the inner surface includes at least one groove formed therein and that extends from the distal terminus at least partially toward the proximal terminus.

2. The aspiration catheter of example 1 wherein the at least one groove extends from the distal terminus to the proximal terminus.

3. The aspiration catheter of example 1 or example 2 wherein the lumen extends along a longitudinal axis, and wherein the at least one groove revolves circumferentially about the longitudinal axis between the proximal terminus and the distal terminus.

4. The aspiration catheter of example 3 wherein the at least one groove revolves about 0.05 times or more per inch about the longitudinal axis between the proximal terminus and the distal terminus.

5. The aspiration catheter of example 3 wherein the at least one groove revolves between about 0.13-0.15 times per inch about the longitudinal axis between the proximal terminus and the distal terminus.

6. The aspiration catheter of example 3 wherein the at least one groove revolves more than about 0.13 times per inch about the longitudinal axis between the proximal terminus and the distal terminus.

7. The aspiration catheter of any one of examples 1-6 wherein the at least one groove has a spiral shape.

8. The aspiration catheter of any one of examples 1-7 wherein the at least one groove comprises a plurality of grooves.

9. The aspiration catheter of example 8 wherein the plurality of grooves are equally spaced apart about a circumference of the inner surface.

10. The aspiration catheter of example 8 or example 9 wherein the lumen extends along a longitudinal axis, and wherein the plurality of grooves revolves circumferentially about the longitudinal axis between the proximal terminus and the distal terminus.

11. The aspiration catheter of example 10 wherein the plurality of grooves revolves about 0.05 times or more per inch about the longitudinal axis between the proximal terminus and the distal terminus.

12. The aspiration catheter of example 10 wherein the plurality of grooves revolves between about 0.13-0.15 times per inch about the longitudinal axis between the proximal terminus and the distal terminus.

13. The aspiration catheter of example 10 wherein the plurality of grooves revolves more than about 0.13 times per inch about the longitudinal axis between the proximal terminus and the distal terminus.

14. The aspiration catheter of any one of examples 1-13 wherein the catheter further comprises:
an inner liner having the inner surface, wherein the at least one groove is formed in the inner liner;
a braid of wires over the inner liner;
a wire coiled over the inner liner; and
an outer sheath over the braid, the wire, and the inner liner.

15. An aspiration catheter, comprising:
a proximal terminus;
a distal terminus; and
an inner surface defining a lumen extending along a longitudinal axis, wherein the inner surface includes a plurality of grooves formed therein, wherein the grooves extend at least partially between the distal terminus and the proximal terminus, and wherein the grooves revolve circumferentially about the longitudinal axis between the distal terminus and the proximal terminus.

16. The aspiration catheter of example 15 wherein the grooves revolve between about 0.13-0.15 times per inch about the longitudinal axis between the proximal terminus and the distal terminus.

17. The aspiration catheter of example 15 or example 16 wherein the grooves are equally spaced about a circumference of the inner surface.

18. The aspiration catheter of any one of examples 15-17 wherein the grooves extend to the distal terminus.

19. The aspiration catheter of any one of examples 15-18 wherein the grooves extend entirely between the distal terminus and proximal terminus.

20. A system for removing material from within a lumen of a human patient, the system comprising:
an aspiration catheter configured to be positioned at a treatment site proximate to the material within the lumen, wherein the aspiration catheter comprises—
   a proximal terminus;
   a distal terminus; and
   an inner surface defining a lumen, wherein the inner surface includes at least one groove formed therein and that extends from the distal terminus at least partially toward the proximal terminus;
a tubing assembly fluidly coupled to the catheter and including a fluid control device; and
a pressure source fluidly coupled to the tubing assembly and configured to generate negative pressure, wherein the fluid control device is movable between (a) a first position in which the pressure source is fluidly connected to the aspiration catheter via the tubing assembly and (b) a second position in which the pressure source is fluidly disconnected from the aspiration catheter.

21. The system of example 20 wherein the lumen extends along a longitudinal axis, wherein the at least one groove includes a plurality of grooves, wherein the grooves are equally spaced about a circumference of the inner surface, wherein the grooves extend at least partially from the distal terminus to the proximal terminus, and wherein the grooves revolve circumferentially about the longitudinal axis between the proximal terminus and the distal terminus.

22. A method for removing material from within a lumen of a human patient, the method comprising:
positioning a distal portion of an aspiration catheter proximate to the material within the lumen, wherein the aspiration catheter includes an inner surface having at least one groove formed therein, and wherein the at least one groove extends from a distal terminus of the aspiration catheter at least partially toward a proximal terminus of the aspiration catheter;
coupling a pressure source to the aspiration catheter via a fluid control device, wherein (a) opening of the fluid control device fluidly connects the pressure source to the aspiration catheter and (b) closing of the fluid control device fluidly disconnects the pressure source from the aspiration catheter;
activating the pressure source to generate a vacuum while the fluid control device is closed; and
opening the fluid control device to apply the vacuum to the aspiration catheter to thereby aspirate at least a portion of the material into the aspiration catheter.

23. The method of example 22 wherein the at least one groove defines a leak path past the clot material when the clot material is aspirated into the aspiration catheter.

24. The method of example 22 or example 23 wherein the at least one groove generates a helical flow pattern in the lumen when the clot material is aspirated into the aspiration catheter.

25. A mandrel for use in forming a catheter, comprising:
a cylindrical body; and
a plurality of features extending radially outward from the body, wherein the catheter is configured to be formed over the body and the features such that the catheter has a plurality of grooves corresponding to an arrangement of the features.

26. The mandrel of example 25 wherein the features are integrally formed with the body.

27. The mandrel of example 25 wherein the features are wires.

28. The mandrel of any one of examples 25-27 wherein the features extend helically about the body.

29. A method of forming a catheter, the method comprising:
positioning an inner liner of the catheter about a mandrel, wherein the mandrel includes a cylindrical body and a plurality of features extending radially outward from the body;
positioning an outer liner of the catheter over the inner liner about the mandrel; and
heating the inner liner and the outer liner such that the inner liner includes a plurality of grooves corresponding to the features of the mandrel.

30. The method of example 29 wherein the method further comprises cooling the inner liner and the outer liner such that the outer liner fuses to the inner liner.

31. The method of example 29 or example 30 wherein the method further comprises rotating the mandrel after heating the inner liner and the outer liner to revolve the features and the corresponding grooves in the inner liner.

32. The method of any one of examples 29-31 wherein the features are integrally formed with the body.

33. The method of any one of examples 29-31 wherein the features are wires.

34. The method of any one of examples 29-33 wherein the features extend helically about the body.

### VII. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An aspiration catheter, comprising:
a proximal terminus;
a distal terminus; and
an inner surface defining a lumen, wherein the inner surface includes at least one groove formed therein and that extends from the distal terminus at least partially toward the proximal terminus.

2. The aspiration catheter of claim 1 wherein the at least one groove extends from the distal terminus to the proximal terminus.

3. The aspiration catheter of claim 1 wherein the lumen extends along a longitudinal axis, and wherein the at least one groove revolves circumferentially about the longitudinal axis between the proximal terminus and the distal terminus.

4. The aspiration catheter of claim 3 wherein the at least one groove revolves about 0.05 times or more per inch about the longitudinal axis between the proximal terminus and the distal terminus.

5. The aspiration catheter of claim 3 wherein the at least one groove revolves between about 0.13-0.15 times per inch about the longitudinal axis between the proximal terminus and the distal terminus.

6. The aspiration catheter of claim 3 wherein the at least one groove revolves more than about 0.13 times per inch about the longitudinal axis between the proximal terminus and the distal terminus.

7. The aspiration catheter of claim 1 wherein the at least one groove has a spiral shape.

8. The aspiration catheter of claim 1 wherein the at least one groove comprises a plurality of grooves.

9. The aspiration catheter of claim 8 wherein the plurality of grooves are equally spaced apart about a circumference of the inner surface.

10. The aspiration catheter of claim 8 wherein the lumen extends along a longitudinal axis, and wherein the plurality of grooves revolves circumferentially about the longitudinal axis between the proximal terminus and the distal terminus.

11. The aspiration catheter of claim 10 wherein the plurality of grooves revolves about 0.05 times or more per inch about the longitudinal axis between the proximal terminus and the distal terminus.

12. The aspiration catheter of claim 10 wherein the plurality of grooves revolves between about 0.13-0.15 times per inch about the longitudinal axis between the proximal terminus and the distal terminus.

13. The aspiration catheter of claim 10 wherein the plurality of grooves revolves more than about 0.13 times per inch about the longitudinal axis between the proximal terminus and the distal terminus.

14. The aspiration catheter of claim 1 wherein the catheter further comprises:
an inner liner having the inner surface, wherein the at least one groove is formed in the inner liner;
a braid of wires over the inner liner;
a wire coiled over the inner liner; and
an outer sheath over the braid, the wire, and the inner liner.

15. An aspiration catheter, comprising:
a proximal terminus;
a distal terminus; and
an inner surface defining a lumen extending along a longitudinal axis, wherein the inner surface includes a plurality of grooves formed therein, wherein the grooves extend at least partially between the distal terminus and the proximal terminus, and wherein the grooves revolve circumferentially about the longitudinal axis between the distal terminus and the proximal terminus.

16. The aspiration catheter of claim 15 wherein the grooves revolve between about 0.13-0.15 times per inch about the longitudinal axis between the proximal terminus and the distal terminus.

17. The aspiration catheter of claim 15 wherein the grooves are equally spaced about a circumference of the inner surface.

18. The aspiration catheter of claim 15 wherein the grooves extend to the distal terminus.

19. The aspiration catheter of claim 15 wherein the grooves extend entirely between the distal terminus and proximal terminus.

20. A system for removing material from within a lumen of a human patient, the system comprising:
an aspiration catheter configured to be positioned at a treatment site proximate to the material within the lumen, wherein the aspiration catheter comprises—
a proximal terminus;
a distal terminus; and
an inner surface defining a lumen, wherein the inner surface includes at least one groove formed therein and that extends from the distal terminus at least partially toward the proximal terminus;
a tubing assembly fluidly coupled to the catheter and including a fluid control device; and
a pressure source fluidly coupled to the tubing assembly and configured to generate negative pressure, wherein the fluid control device is movable between (a) a first position in which the pressure source is fluidly connected to the aspiration catheter via the tubing assembly and (b) a second position in which the pressure source is fluidly disconnected from the aspiration catheter.

21. The system of claim 20 wherein the lumen extends along a longitudinal axis, wherein the at least one groove includes a plurality of grooves, wherein the grooves are equally spaced about a circumference of the inner surface, wherein the grooves extend at least partially from the distal terminus to the proximal terminus, and wherein the grooves revolve circumferentially about the longitudinal axis between the proximal terminus and the distal terminus.

22. A method for removing material from within a lumen of a human patient, the method comprising:
positioning a distal portion of an aspiration catheter proximate to the material within the lumen, wherein the aspiration catheter includes an inner surface having at least one groove formed therein, and wherein the at least one groove extends from a distal terminus of the aspiration catheter at least partially toward a proximal terminus of the aspiration catheter;
coupling a pressure source to the aspiration catheter via a fluid control device, wherein (a) opening of the fluid control device fluidly connects the pressure source to the aspiration catheter and (b) closing of the fluid control device fluidly disconnects the pressure source from the aspiration catheter;
activating the pressure source to generate a vacuum while the fluid control device is closed; and
opening the fluid control device to apply the vacuum to the aspiration catheter to thereby aspirate at least a portion of the material into the aspiration catheter.

23. The method of claim 22 wherein the at least one groove defines a leak path past the clot material when the clot material is aspirated into the aspiration catheter.

24. The method of claim 22 wherein the at least one groove generates a helical flow pattern in the lumen when the clot material is aspirated into the aspiration catheter.
